(19) United States
(12) Patent Application Publication
(10) Pub. No.: US 2004/0152733 A1
(43) Pub. Date: Aug. 5, 2004

(54) Duloxetine for treatment of hot flashes

(76) Inventors: Owen Brendan Wallace, Zionsville, IN (US); Timothy John Garnett, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/473,634

(22) PCT Filed: Mar. 15, 2002
(86) PCT No.: PCT/US02/05113

(51) Int. Cl. A61K 31/452; A61K 31/381

(52) U.S. Cl. 514/320; 514/438

(57) ABSTRACT

The present invention provides methods for treatment of hot flashes in a mammal by administering duloxetine to that mammal. Another aspect of the invention is a method for treatment of hot flashes in a human female undergoing raloxifine adminstration by administration of duloxetine to that female. Another aspect of the invention is a method of treating hot flashes in a human undergoing estrogen replacement therapy comprising administering an effective amount of duloxetine.

### FIELD OF THE INVENTION

The invention relates to a method for using duloxetine for the treatment of hot flashes.

### BACKGROUND OF THE INVENTION

Hot flashes or flushing is characterized by a sudden onset of warmth in the face and neck and often progressing to the chest. Such an episode generally lasts several minutes and is evidenced by a visible flushing of the skin. Often such episodes are accompanied by sweating, dizziness, nausea, palpitations and diaphoresis. Such symptoms can disrupt sleep and interfere with the quality of life.

In general, menopause is associated with vasomotor symptoms, manifested by hot flashes, which are variable in frequency and severity, and may persist for several months or a few years. Approximately 75% of menopausal women will experience hot flashes during menopause (McKinlay, S., Jeffreys, M., “The Menopausal Syndrome,” J. Prev. Soc. Med., 28:108, 1974), with 80% experiencing them for greater than one year and 25 to 50% for greater than 5 years. Judd, H. L., Cleary, R. E., Creasman, W. T., et al., “Estrogen Replacement Therapy,” Obstet. Gynecol., 58-267, 1981. For some of these women, the symptoms are disabling. Gambrell, R. D., Jr. “The Menopause: Benefits and Risks of Estrogen-Progestogen Replacement Therapy,” Fertil. Steril., 37:457, 1982. The standard therapy for alleviating these symptoms is estrogen replacement therapy (ERT). Many women, unfortunately, are not candidates for ERT because such therapy is medically contraindicated (e.g., estrogen sensitive carcinoma and thromboembolic disease). Furthermore, this therapy, while effective, suffers from poor patient compliance, due to unpleasant side-effects, poor oral absorption, and poor bio-availability of the natural estrogens 17β-estradiol and estrone.

Men may also have hot flashes following androgen-deprivation therapy (from bilateral orchiectomy or treatment with a gonadotrophin-releasing-hormone agonist) for metastatic prostate cancer.

Non-hormonal alternatives for hot-flashes are extremely limited at present and have been associated with poor response in many patients. The two most widely used no-hormonal therapeutic modalities at present in the United States are clonidine and Bellargal spacetabs. Neither has gained wide clinical acceptance because of poor effectiveness and side effects.

A recent report has established in a pilot study that 45% of venlafaxine treated menopausal women (survivors of breast cancer) suffering from hot flashes reported a greater than 50% decrease in hot flash frequency versus only 20% in treatment with placebo. “Venlafaxine in management of hot flashes in survivors of breast cancer: a randomised controlled trial,” The Lancet (2000), 356(9247), 2059-2063.

The investigators hypothesized that the hot flash activity may be alleviated via treatment with venlafaxine. However, over 50% of venlafaxine treated menopausal women did not report a 50% decrease in hot flash frequency. Id. at 2059. In addition, the investigators noted that any venlafaxine “efficacy must be balanced against the drug's side-effects.” Thus, although the hot flash mechanism may indeed be mediated through serotonin and norepinephrine reuptake inhibition, it can not be predicted a priori whether a pharmaceutical that is classified as a SSRI is effective at decreasing the incidence of hot flashes. Further, it would be optimal to find a method of treatment for hot flashes with greater efficacy and/or greater safety.

### BRIEF SUMMARY OF THE INVENTION

In accordance with the present invention, there is provided a method of treating hot flashes in a mammal comprising the administration to a patient in need of such treatment an effective amount of duloxetine.

Another aspect of the invention is a method for treating hot flashes in a human female undergoing ERT comprising administering duloxetine to a human female in need thereof an effective amount of duloxetine.

Another aspect of the invention is a method for treating hot flashes comprising administering duloxetine to a human female where estrogen replacement thereof is contradicted.

Another aspect of the invention is a method for treating hot flashes in a human female undergoing raloxifene administration comprising administering duloxetine to a human female in need thereof an effective amount of duloxetine.

Further aspects of the present invention include a use of duloxetine for the manufacture of a medicament for treating hot flashes in a human, use of duloxetine for the manufacture of a medicament for treating hot flashes in a human undergoing ERT and a use of duloxetine for the manufacture of a medicament for treating hot flashes in a human female undergoing raloxifene administration.

### DETAILED DESCRIPTION AND PREFERRED EMBODIMENTS

Duloxetine is N-methyl-3-(1-naphthalenyloxy)-3-(2-thienyl)propanamine. It is usually administered as the (+) enantiomer, and as the hydrochloride salt. It was first taught by U.S. Pat. No. 4,956,388, which teaches the synthesis of the compound as well as its high potency as an uptake inhibitor of both serotonin and norepinephrine. The word “duloxetine” will be used here to refer to any acid addition salt or the free base of the molecule, as well as to either an enantiomer or the racemate. It is to be understood, however, that the (+) enantiomer is preferred.

As used herein, the term “active ingredient” refers to duloxetine as it is usually administered.

The term “treating” (or “treat”) as used herein includes its generally accepted meaning which encompasses prohibiting, preventing, restraining, and slowing, stopping, decreasing the incidences or reversing progression, severity, of a resultant symptom. As such, the methods of this invention encompass both therapeutic and prophylactic administration.

As used herein the term “effective amount” refers to the amount or dose of the compound, upon single or multiple dose administration to the patient, which provides the desired effect in the patient under diagnosis or treatment.

An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances. For example, a typical daily dose may contain from about 60 to about 80 mg of the active ingredient. The compounds can be administered by a variety of routes including oral, rectal, transdermal, subcutaneous, intravenous, intramuscular, bucal or intranasal routes. Alternatively, the compound may be administered by continuous infusion.

As used herein the term “patient” refers to a mammal, such as a mouse, guinea pig, rat, dog or human. It is understood that the preferred patient is a human.

The compound is particularly selective, having few if any physiological effects besides those on norepinephrine and serotonin processing, and therefore is free of side effects and unwanted activities. Further, it is effective at relatively low doses, as discussed below, and may safely and effectively be administered once or twice per day. Thus, difficulties created by the multiple dosing of patients, who are children and disorganized adults, are completely avoided.

The most preferred dose of duloxetine for the treatment of a given patient with any particular disorder will vary, depending on the characteristics of the patient, as all clinicians and medical doctors are aware. Factors such as other diseases from which the patient suffers, the patient's age and size, and other medications which the patient may be using will have an effect on the duloxetine dose and will be taken into account. In general, however, the daily dose of duloxetine is from about 1 to about 80 mg. A more preferred dose range is from about 60 to about 80 mg daily.

Another preferred dose is about 60 mg taken once per day. Another preferred dose is 40 mg taken twice per day.

Duloxetine is orally available and presently is orally administered, in the form of a tablet or a capsule full of enteric coated granules. Oral administration in such forms is preferred in the practice of the present invention. However, other routes of administration are also practical and may be preferred in certain cases. For example, transdermal administration may be very desirable for patients who are forgetful or petulant about taking oral medicine. Sustained release formulations, oral or percutaneous, may be prepared, but are not preferred because duloxetine is quite effective when administered once or twice daily and there is little benefit from the additional effort of preparing the sustained action product.

In general, the formulation of duloxetine for use in the present invention follows the methods used in formulating duloxetine for other purposes, and indeed methods usual in pharmaceutical science are appropriate. However, a preferred formulation of duloxetine comprises enteric pellets, or granules, of which a number are charged in a gelatin capsule.

Raloxifene hydrochloride (raloxifene) is described in U.S. Pat. No. 4,418,068 and is known to be effective in treating the symptoms of post menopausal syndrome, particularly osteoporosis. Indeed, raloxifene was approved for marketing as a preventive agent for osteoporosis by the U.S. Food and Drug Administration in late 1997. Raloxifene has the following structure:

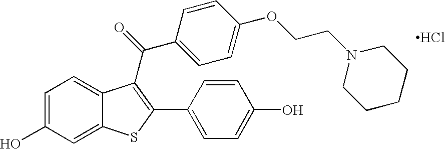

Clinical studies of raloxifene demonstrated a slight increase in the number of women, relative to placebo, who reported incidences of hot flashes during the clinical trial. (24.6% for raloxifene vs. 18.3% for placebo).

The preferred duloxetine enteric formulation comprises a) a core consisting of duloxetine and a pharmaceutically acceptable excipient; b) an optional separating layer; c) an enteric layer comprising hydroxypropylmethylcellulose acetate succinate (HPMCAS) and a pharmaceutically acceptable excipient; d) an optional finishing layer.

The duloxetine layer was built up by suspending duloxetine in a 4% w/w solution of the hydroxypropylmethyl-cellulose in water, and milling the suspension with a CoBall Mill (Fryma Mashinen AG, Rheinfelden, Switzerland) model MS-12. A fluid bed dryer with a Wurster column was used to make this product, at a batch size of 1.0 kg. The separating layer was added from a 4% w/w solution of the hydroxypropyl-methylcellulose in water, in which the sucrose was also dissolved.

In order to prepare the enteric coating suspension, purified water was cooled to 10° C. and the polysorbate, triethyl citrate and silicone emulsion were added and dispersed or dissolved. Then the HPMCAS and talc were added and agitated until homogeneity was obtained, and the HPMCAS was fully neutralized by addition of ammonium hydroxide until solution of the polymer was completes To this suspension, a carboxymethylcellulose aqueous solution, 0.5% w/w, was added and blended thoroughly. The enteric suspension was maintained at 20° C. during the coating process. The enteric suspension was then added to the partially completed pellets in the Wurster column at a spray rate of about 15 ml/min, holding the temperature of the inlet air at about 50° C. The product was dried in the Wurster at 50° C. when the enteric suspension had been fully added, and then dried on trays for 3 hours in a dry house at 60° C. A finishing layer was then applied which consisted of a 4.5% w/w/hydroxypropylmethyl-cellulose solution containing titanium dioxide and propylene glycol as plasticizer. The pellets were completely dried in the fluid bed dryer and then were then filled in size 3 gelatin capsules.

When duloxetine and raloxifene are both employed, they may be administered sequentially, concurrently, or simultaneously as a single composition to the subject. If administered sequentially, the period between the administration of duloxetine and raloxifene will typically be one week to one month, and optimally, one day to one week. In a preferred administration scheme, the human will receive duloxetine and raloxifene concurrently or simultaneously.

In accordance with one method of use, duloxetine and raloxifene may be administered systemically orally.

The precise dosage necessary will vary with the age, size, sex and condition of the subject, the nature and severity of the disorder to be treated, and the like; thus, a precise effective amount should be determined by the caregiver. In general terms, an effective dose of duloxetine will range between values described above.

When duloxetine is administered with raloxifene, the total dosage (per day) of raloxifene will typically be in the range from about 1 mg to 1000 mg per day, usually being in the range from about 10 mg to 100 mg per day, preferably being in the range from about 25 mg to 75 mg per day, more preferably being in the range from about 55 mg to 65 mg per day, and most preferably being 60 mg per day.

### EXAMPLE 1

The patient to be benefited by practice of the present invention is a patient experiencing vasomotor symptoms such as hot flashes, a sudden brief flushing and sensation of heat caused by dilation of skin capillaries. Diagnosis of this disorder is to be made by a physician. It is presently believed that duloxetine's potency in inhibiting the uptake of serotonin and norepinephrine is the mechanism by which it benefits such patients, by alleviating the effects of the disorder from which the patient suffers, or even eliminating the disorder completely.

The following description is put forth so as to provide those of ordinary skill in the art with a complete disclosure and description of the effectiveness of the compositions and methods of the invention and are not intended to limit the scope of what the inventors regard as their invention.

Patients eligible for a clinical trial include women who are either 1) naturally menopausal; or 2) pre-menopausal but had undergone bilateral oophorectomy surgery within four weeks prior to the commencement of the study. All the women in the study experience a minimum of thirty five hot flashes per week. Men considered for a clinical trial would have androgen deprivation therapy for prostate cancer scheduled to continue at least 6 weeks beyond the trial entry date. Men have bothersome hot flashes for at least the previous month a minimum of fourteen times weekly of sufficient severity to desire therapeutic intervention. The women or men are divided into two groups for a randomized double-blind placebo controlled study.

The groups receive drug or placebo as illustrated below:

Group 1: Duloxetine (60 mg QD)+Placebo

Group 2: Placebo+Placebo

Group 3: Raloxifene (60 mg QD)+Placebo

Group 4: Raloxifene (50 mg QD)+Duloxetine (30 mg QD)

For three weeks both groups are administered placebo only. For eight to twelve weeks thereafter, each group is administered drug or placebo. Data is collected (numbers/severity of hot flashes experienced) from each participant during and at the end of the test period.

The treatment of the clinical trial participants with duloxetine results in a decrease, relative to the placebo groups (Groups 2 and 3), of the incidence of hot flashes in the duloxetine only group (Group 1) and the duloxetine/raloxifene group (Group 4). This decrease indicates the utility of the invention.

The invention has been described with reference to the preferred embodiment. Obviously, modifications and alterations will occur to others upon a reading and understanding of this specification. It is intended to include all such modifications and alterations insofar as they come within the scope of the appended claims or the equivalents thereof.

We claim:

1. A method of treating hot flashes in a mammal comprising administering to a mammal in need thereof an effective amount of duloxetine.

2. A method as claimed in claim 1 wherein the mammal is administered between 30 and 150 mg of duloxetine per day.

3. A method as claimed in claim 1 wherein the mammal is administered between 40 and 80 mg of duloxetine per day.

4. A method as claimed in claim 2 wherein the duloxetine is administered as an enteric capsule.

5. Duloxetine for use in the treatment of hot flashes.

6. A pharmaceutical formulation containing, as an active ingredient, duloxetine adapted for use in the treatment of hot flashes.

7. The use of duloxetine for the manufacture of a medicament for the treatment of hot flashes.

8. A method of treating hot flashes in a human undergoing raloxifene administration comprising administering an effective amount of duloxetine to a human in need thereof.

9. The method according to claim 7 where the raloxifene is raloxifene hydrochloride.

10. The method according to claim 7 where the administration of duloxetine and raloxifene is concurrent.

11. The method according to claim 9 where the administration of duloxetine and raloxifene is simultaneous.

12. The use of duloxetine for the manufacture of a medicament for treating hot flashes in a human female undergoing raloxifene administration.

13. A pharmaceutical formulation adopted for treatment of hot flashes in humans comprising duloxetine and raloxifene.

14. A method of treating hot flashes in a human undergoing estrogen replacement therapy comprising administering an effective amount of duloxetine to a human in need thereof.

15. The method according to claim 13 where the administration of duloxetine and estrogen replacement therapy are concurrent.

16. The method according to claim 14 where the administration of duloxetine and estrogen replacement therapy are simultaneous.

17. The use of duloxetine for the manufacture of a medicament for treatment of hot flashes in a human female undergoing estrogen replacement therapy.

18. A pharmaceutical formulation adopted for treatment of hot flashes in humans comprising duloxetine and estrogen replacement therapy.

19. A method of treating hot flashes in a human female comprising administering to a woman in need thereof an effective amount of duloxetine estrogen replacement therapy is contraindicated.

20. The use of duloxetine for the manufacture of a medicament for treatment hot flashes in a human female where estrogen replacement therapy is contraindicated.
